(19) United States
(12) Patent Application Publication
(10) Pub. No.: US 2018/0353173 A1
(43) Pub. Date: Dec. 13, 2018

(54) COATED SUTURE SYSTEM FOR HEALING AUGMENTATION OF SOFT TISSUE REPAIR AND RECONSTRUCTION

(71) Applicant: Ramsey KINNEY, Orlando, FL (US)

(72) Inventor: Ramsey KINNEY, Orlando, FL (US)

(21) Appl. No.: 15/781,338

(22) PCT Filed: Dec. 5, 2016
(86) PCT No.: PCT/US2016/065021

§ 371 Date: Jun. 4, 2018

Related U.S. Application Data
(60) Provisional application No. 62/262,854, filed on Dec. 3, 2015.

(51) Int. Cl. A61B 17/06; B05D 1/28

(52) U.S. Cl. CPC A61B 17/06166; A61B 2017/00526; B05D 1/28

(57) ABSTRACT

Disclosed herein are systems, apparatuses and methods for applying commercially available sutures or other wound repair devices to improve and/or accelerate healing. In an exemplified embodiment, provided are methods, apparatuses, and systems that enable intra-operative application of wound repair devices with biologic and/or pharmaceutical compositions to facilitate controlled delivery of bioactive molecules, resulting in improved musculoskeletal soft tissue repair and reconstruction. In a specific embodiment, a method including obtaining an isolated platelet rich plasma (PRP) sample, and applying the sample to a suture under conditions to provide a dry coat of PRP onto the suture is disclosed herein.

### BACKGROUND

Orthopedic surgeons perform a variety of surgical procedures to repair and reconstruct soft tissue pathologies. These encompass acute traumatic injuries such as ligament ruptures and tendon lacerations, but also chronic degenerative conditions including various tendinopathies. Tendon and ligaments have relatively poor vascularity, and therefore have decreased and delayed healing potential. The rehabilitation of patients after surgery must be limited and carefully controlled to allow healing of the tissues and avoid failure of the repair or reconstruction.

Platelet rich plasma (PRP) is a component of a whole blood that is rich in growth factors. In order to obtain this component of the blood, blood is centrifuged initially to remove a majority of the cellular component that is comprised of erythrocytes and leukocytes. The remaining plasma is then centrifuged again to concentrate the platelets and growth factors. PRP is currently being used as an injectable therapy to treat tendonitis, arthritis, and various other degenerative musculoskeletal pathologies. Animal studies have shown benefits of PRP therapies for soft tissue injuries. However, there is no standardized method of preparation and delivery of PRP to a patient in need.

### SUMMARY

Disclosed herein are methods, apparatuses and systems that provide wound repair devices having improved characteristics to facilitate healing or other related medical outcomes. In an embodiment, disclosed is a method that involves obtaining an isolated platelet rich plasma (PRP) sample, and applying the sample to a wound repair device under conditions to produce a dry coat of PRP onto the suture is provided. In a specific example, the method provides a quick and efficient way to dry coat a suture in an operating room using a patient's own PRP.

In another embodiment, a system for applying a composition to a wound repair device for tissue repair is provided. The composition, may for example, include a biologic, a pharmaceutical composition, or a combination thereof. The system includes at least a first set of coating rollers including at least a first roller and a second roller, configured to receive a wound repair device there between, the first and second roller configured to rotate in opposing directions as the wound repair device is passed there between, wherein the first and/or second roller includes a composition, such that when the wound repair device is passed between the first and second rollers, the composition is applied to the wound repair device. The system includes at least a first guide roller configured to rotate and guide the wound repair device to allow the composition to adhere onto the wound repair device, such that when the wound repair device is guided by the third roller, the composition is dried thereon.

In a further embodiment, an intra-operative apparatus for coating a wound repair device is provided herein. The apparatus includes at least one coating chamber; and at least one drying chamber, wherein the coating chamber is configured to contain a composition to be coated on a wound repair device inserted therein, and the drying chamber is configured to dry the composition-coated wound repair device. The composition may include a biologic, a pharmaceutical composition, or a combination thereof. The apparatus may include a first connector port in communication with the coating chamber, wherein a composition may be introduced through the first connector port. The first connector port may be adapted to receive a syringe, said syringe comprising the composition to be introduced into the coating chamber. A second connector port may be in communication with the drying chamber, the second connector port configured to connect to a suction device or an airflow device, wherein the suction or airflow device is configured to provide a movement of air in the drying chamber. The suction device may provide a convective flow of air in the drying chamber when associated therewith to dry the composition on the wound repair device. The airflow device may deliver air to the drying chamber to dry the composition on the wound repair device. The wound repair device may be rotated between the coating chamber and the drying chamber in some non-limiting embodiments.

### BRIEF DESCRIPTION OF THE DRAWINGS

FIG. 1 includes a schematic drawing of an embodiment of the invention described herein.

FIG. 2 includes a perspective view of an embodiment of the invention described herein.

FIG. 3 includes a top plan view of the embodiment of the invention shown in FIG. 2.

FIG. 4 is a perspective view of an embodiment of an intra-operative apparatus.

FIG. 5 is a perspective view of the apparatus embodiment of FIG. 4, wherein a frame of the apparatus has been adjusted such that wound repair device can be inserted into a coating chamber.

FIG. 6 is a perspective view of the apparatus embodiment of FIG. 4, wherein the frame of the apparatus has been adjusted such that the wound repair device can be inserted into a drying chamber.

FIG. 7 is a top side view of the apparatus embodiment shown in FIG. 4

FIG. 8 includes a graphical representation of total protein content of each sample compared to the number of coating cycles.

FIG. 9 includes a graphical representation demonstrating tensile load to failure showed no significant difference with relation to the number of PRP coatings.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated apparatus, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. It is to be noted that all ranges disclosed within this specification are inclusive and are independently combinable.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order, quantity or importance, but rather the terms first, second, etc., are used to distinguish one element from another.

Definitions:

The term “platelet rich plasma” as used herein includes, but is not limited to a component of a whole blood that is rich in growth factors. The term “isolated platelet rich plasma” indicates the component of whole blood rich in growth factors has been at least partially separated from other components of the blood, including erythrocytes and leukocytes. This may occur by way of centrifugation of a blood sample, in order to remove a majority of the cellular component comprised of non-platelet rich plasma, non-PRP.

The term “allogenic” as used herein, includes, but is not limited to denoting, relating to or involving tissues or cells that are genetically dissimilar and may be immunologically incompatible, but are from individuals of the same species.

The term “xenogenic” as used herein, includes, but is not limited to originating outside of the organism, or from a foreign substance that has been introduced into the organism.

The term “autogenic” as used herein, includes, but is not limited to produced from within the subject. In a non-limiting example, a biologic that is autogenic, is one that is taken from a subject, and then used to repair tissue within the same subject.

As used herein, the terms “subject”, “user” and “patient” are used interchangeably. As used herein, the term “subject” refers to an animal, preferably a mammal such as a non-primate (e.g., cows, pigs, horses, cats, dogs, rats etc.) and a primate (e.g., monkey and human). In some non-limiting embodiments, the subject is a human suffering from tissue damage or undergoing a procedure for tissue repair.

As used herein, the term “biologic” refers to, but is not limited to, an agent composed of sugars, proteins, lipids or nucleic acids or complex combinations of these substances, or may be living entities such as cells and tissues. Biologics are either isolated from a variety of natural sources—human, animal, or microorganism—or may be synthesized by biotechnology methods. In one, non-limiting example, the biologic may include a platelet rich plasma sample. PRP may be supplemented with antibodies, growth factors, or hormones or other biologics to supplement PRP or plasma.

As used herein, the term “pharmaceutical composition” includes, but is not limited to, a composition including one or more compounds which may be applied onto a wound repair device as described herein, in non-limiting embodiments, to a suture, instead of or in addition to a biologic applied thereon. Non-limiting examples of pharmaceutical compositions include: antibiotics, chemotherapeutic agents, analgesics/anti-inflammatories, anti-coagulants, coagulants, vasodilators, vasoconstrictors, or bioactive small molecules. Chemotherapeutics may be used for focal treatment after a tumor resection, for example. Antibiotics may be useful for the treatment or prevention of an infection at or near the surgical site or a site of trauma, for example. Analgesics and anti-inflammatory may be used to localize pain control at the site of surgery. Coagulants may improve localized control of bleeding, and anti-coagulants may provide localized anti-coagulation to prevent clotting, for example in vascular repair procedures. Vasodilators may be used to improve microvascular blood flow at the surgical site to promote healing, and vasoconstrictors may be used to decrease microvascular blood flow to decrease bleeding at the surgical site. Bioactive small molecules may provide local delivery of small molecules via the embodiments described herein to promote biologic activity. In a specific example, the combination of L-ascorbic acid 2-phosphate, beta-glycerophosphate, and dexamethasone is applied to a wound repair device.

The term “wound repair device” as used herein, includes but is not limited to a component that may contact, either directly or indirectly, a damaged tissue, or a tissue requiring repair or healing, in non-limiting examples. In one non-limiting embodiment, wound repair device includes sutures. In other non-limiting embodiments, wound repair device may include other geometries such as bandages, sponges, beads or patches, for example.

The term “coating” or “coated” as used herein, for example, in reference to applying a composition to a wound repair device by coating the composition with the wound repair device, refers to but is not limited to contacting the wound repair device with the composition. The terms “coating” or “coated” does not require the wound repair device be covered completely in the composition. In some embodiments, only a portion of the wound repair device may be coated with the composition, i.e., the wound repair device may be partially coated in a non-limiting example. In one example, “coating” may include dipping the wound repair device into the composition such that at least some portion of the composition adheres onto the wound repair device. In another example, “coating” may include painting the composition onto the wound repair device using a brush or a roller, in non-limiting embodiments. In still another non-limiting embodiment, “coating” may include stamping the composition onto the wound repair device, for example, when the wound repair device comprises a patch.

The term “associating” or “associated” as used herein, for example, may describe a relationship of components of the system or apparatus embodiments described herein. The term associating or associated includes but is not limited to direct and indirect attachment with, adjacent to, in contact with, partially or fully attached to, and/or in close proximity therewith. In one non-limiting example, “associated” describes the relationship between the vacuum and other components of the system to dry the composition onto the wound repair device.

### DESCRIPTION OF EMBODIMENTS

Delivery of platelet rich plasma (PRP) or other biologic during repair or reconstruction of a musculoskeletal injury may enhance the healing process, accelerate recovery and shorten rehabilitation. A patient who is provided PRP would be able to return to work or sports quicker and with decreased risk of re-injury.

The inventors have developed systems, apparatuses and methods for applying commercially available sutures or other wound repair devices to improve and/or accelerate healing. In a specific embodiment, provided are methods, apparatuses, and systems that enable intra-operative application of wound repair devices with biologic and/or pharmaceutical compositions to facilitate controlled delivery of bioactive molecules, resulting in improved musculoskeletal soft tissue repair and reconstruction. There are multiple design considerations that need to be addressed for integration into the sterile operating room (OR) environment. Accordingly, embodiments involving application of biologics and/or pharmaceutical compositions are efficient to avoid increasing surgical time. Apparatuses can be of various sizes, and may be portable to be used on a surgical table in some non-limiting embodiments. Certain embodiments proposed herein may include designs for an apparatus and/or system which can be manufactured inexpensively and may be disposable in some non-limiting embodiments. The apparatus and/or system may be easily sterilized with steam autoclave so that it is reusable and cost effective, in other non-limiting embodiments. The embodiments described herein may make use of utilities already available in the OR like vacuum and compressed air capabilities. The embodiments described herein serve to ameliorate denaturing of proteins in the compositions being applied to materials in some non-limiting embodiments.

The inventors have discovered a system, apparatus, and a method of delivery of PRP with the use of sutures. This system, apparatus and method could be employed during a surgical procedure and results in an increase in successful post-surgery results, quicker recovery, and a more complete recovery for patients. Sutures are already used in surgical repairs and reconstruction to reattach injured tissues or secure grafts to muscle and bone. The system and method embodiments described herein may be used to weave a coated suture through tissue such as a reconstructed ligament or repaired tendon, to optimize the gradient of growth factors at that location and the surrounding area, and to promote the migration of cells into the tissue for quicker and more effective healing.

In one non-limiting embodiment, the system for applying a composition (i.e., a biologic and/or a pharmaceutical composition) to a wound repair device for tissue repair may include a housing and one or more coating rollers associated with one another, held within the housing, and configured to receive a wound repair device between the rollers. The coating rollers may include a composition as described herein for coating the wound repair device, in a non-limiting embodiment. One or more guide rollers may also be included, wherein the wound repair device may be passed through the guide roller(s) to allow the composition to dry onto the wound repair device as it passes through the system, in one embodiment. Additional non-limiting embodiments may include a vacuum, a suction hose, or other drying apparatus to facilitate more rapid drying of the composition onto the wound repair device.

The system and method embodiments described herein may be readily integrated into the sterile operating room (OR) environment. Many components required for the system and method embodiments described herein are routinely used in the OR for different purposes and in different arrangements. Therefore, many of the utilities required for the operation and ease of integration of the system and method embodiments described herein into the OR are already in place, such as, for example, a vacuum, and compressed air capabilities.

In one embodiment, the method and system herein may be used to alter commercially available wound repair devices, i.e., sutures for use. This alteration may be accomplished in the OR immediately preceding, or during a surgical procedure in which the equipment is needed. The process is efficient, and does not increase surgical time. The system described herein may be a disposable system in some non-limiting embodiments and/or may be a system that can be disinfected and sterilized between uses. The system may be sterilized in a steam autoclave, in one non-limiting embodiment, providing a reusable cost-effective system.

The inventive embodiments provided herein overcome inferior prior art methods which involve heat. Heat causes denaturation of proteins. The inventive embodiments described herein solve the problems of the prior art, and further, minimize loss of biologic agent in the process. The embodiments described herein include a system for coating sutures, providing intuitive handling, and as mentioned, minimizing the loss of biologic agent applied to the sutures.

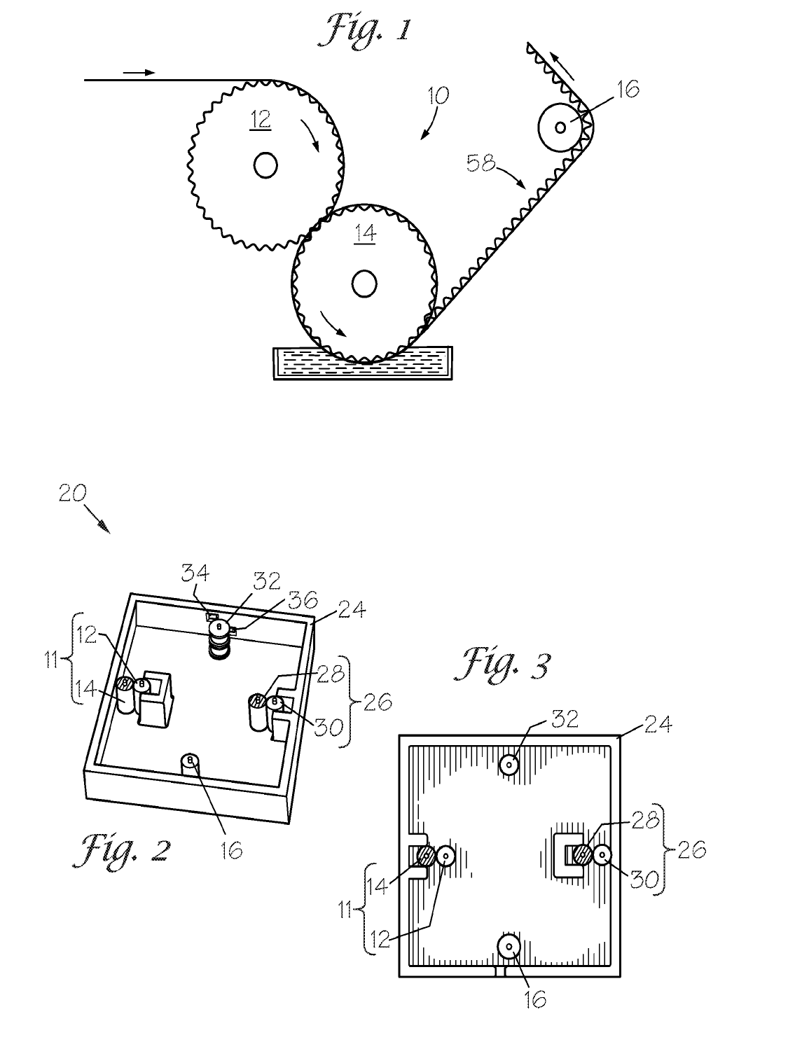

In one, non-limiting embodiment, the system includes a vacuum method of drying the sutures during or following the coating of the sutures. In another non limiting aspect, a gravure coating mechanism, as shown in FIG. 1, is used to guide the sutures being coated via multiple rollers, as the sutures dry in a chamber, in some non-limiting-embodiments, the chamber may be a vacuum chamber. In another non-limiting embodiment, movement of the sutures through the system described herein may provide a coating to both sides of the suture. and may dry each side of the suture between coatings.

As aforementioned, the inventors have discovered a method for delivering a composition (i.e., PRP, pharmaceutical composition, plasma) to a tissue prior to and during the healing process in order to accelerate recovery and shorten rehabilitation. The method includes applying the composition to a wound repair device that will be closely associated with, or placed into, the affected tissue. One embodiment described herein includes the coating of sutures which would be used in the affected tissue to speed up the recovery process. These sutures would be coated with a composition, which may include PRP, in some instances. The coated suture may then be used in the repair or reconstruction to sew the injured tissues together or secure grafts to muscle and/or bone. The coated suture could also be woven through a reconstructed ligament, for example, or a repaired tendon, to optimize the gradient of growth factors and to promote migration of cells into the tissue. In order to do so, the coating suture process may be accomplished, in one embodiment, in the sterile OR environment.

The inventors have discovered that the amount of composition (biologic, or protein, or pharmaceutical composition), for example, applied to the sutures increased with each sequential coating and drying cycle of the suture. The inventors also discovered that following use of the system embodiments described herein, passage of the suture through a tissue following the coating procedure did not strip the coating from the suture.

Embodiments described herein include a system for applying a composition (i.e., a biologic and/or a pharmaceutical composition) to a wound repair device for tissue repair. In one non-limiting embodiment, the wound repair device may include sutures. The system may include at least a first set of coating rollers, wherein a first roller and a second roller are associated with one another, and are configured to receive the wound repair device there between. The first and second rollers rotate in opposing directions as the wound repair device is passed there through. At least one of the rollers has a composition, such that when the wound repair device passes through the first set of coating rollers, at least one side of the wound repair device is coated with the composition. In one non-limiting embodiment, the composition may include a biologic, and the biologic may include PRP. A first guide roller may be provided, wherein following passage through the first set of coating rollers, the wound repair device is passed around a first guide roller to allow the coating to dry onto the wound repair device.

A second set of coating rollers may be provided, which may include a first roller and a second roller, wherein the second roller may include a composition. Once the wound repair device is passed around the first guide roller, it may be passed through the second set of coating rollers wherein the second side of the wound repair device may be coated in the composition. A second guide roller may provide drying of the second side of the wound repair device as the wound repair device passes around the second guide roller. In another non-limiting embodiment, at least one of the first and second set of coating rollers may include a biologic, and the other set of the first and second coating rollers may include a pharmaceutical composition, such that the wound repair device may be coated with a biologic on one portion and a pharmaceutical composition on another portion thereof.

In another, non-limiting embodiment, both of the first set of coating rollers may include a composition, such that both sides of the wound repair device may be coated with the composition as the wound repair device passes through the first set of rollers. In a non-limiting embodiment, both of the second set of rollers encountered by the wound repair device may not include a composition, and may be used to dry the composition on both sides of the wound repair device. In still another embodiment, in those non-limiting examples provided herein, possibly only one side of the wound repair device may be coated in the composition.

In another non-limiting embodiment, an intra-operative apparatus for coating a wound repair device is provided including at least one coating chamber and at least one drying chamber, wherein the coating chamber is configured to contain a composition to be coated on a wound repair device inserted therein, and the drying chamber is configured to dry the composition-coated wound repair device. In another embodiment, the intra-operative apparatus includes a first connector port adjacent to the coating chamber, wherein a composition may be introduced through the first connector port, and a second connector port adjacent to the drying chamber, the second connector port configured to connect to a suction device or an airflow device, wherein the suction or airflow device is configured to provide a movement of air in the drying chamber. The first connector port may be adapted to receive a syringe including the composition to be introduced into the coating chamber.

In one embodiment, a convective air flow may be provided in the drying chamber, and may dry the composition onto the wound repair device. In a particular embodiment, the second connector port may receive a suction device which may cause a convective flow of air in the drying chamber to pass over the wound repair device. In one non-limiting embodiment, the convective flow may move from the top to the bottom of the chamber to accelerate drying of the composition on the wound repair device.

In an alternative embodiment, the second connector port may be associated with an airflow device to deliver air to the drying chamber to dry the composition on the wound repair device. In still another embodiment, the wound repair device may be moved from the coating chamber to the drying chamber and back to the coating chamber for another coat or layer of composition, and back to the drying chamber, any number of times to apply multiple layers or coats of composition onto the wound repair device.

As aforementioned, in non-limiting embodiments, the wound repair device may include various geometries including sutures, beads, or patches, or any other type of wound repair device useful in an intra-operative environment, or in a pre- or post-operative environment, for example. The wound repair device may include geometries including bandages, or components of permanently placed devices, such as plates or screws, for example.

In a further embodiment, a chamber cover is rotatable between the coating chamber and the drying chamber of the intra-operative apparatus, wherein the chamber cover is provided to cover over the chamber that is not in use. Therefore, when the wound repair device to be coated is placed in the coating chamber, the drying chamber is covered with the chamber cover. To dry the composition-coated wound repair device, the wound repair device is removed from the coating chamber, the chamber cover is rotated around toward the coating chamber, and the wound repair device is placed into the drying chamber. A frame may be provided, and may be associated with the apparatus, wherein the wound repair device to be coated and dried may be held by the frame. This would reduce or entirely remove the requirement of handling the sutures or other sterile wound repair device by a user of the apparatus, maintaining sterility of the wound repair device during the coating procedure, in one non-limiting embodiment. In one non-limiting embodiment, the frame may be manipulated by a user during the coating and drying of the composition onto the wound repair device, to move the wound repair device between the coating chamber and the drying chamber.

This apparatus and method provides rapid coating of composition onto a wound repair device, and can provide for rapid layered coatings of composition to be applied to a wound repair device. The inventive embodiments provided herein are useful, for example, during a procedure in which the composition is obtained from a patient during the procedure, and the wound repair device is rapidly coated with the composition using the methods and apparatus described herein during the procedure such that the wound repair device can be used on the patient before completion of the procedure in a non-limiting embodiment.

Osteogenic differentiation is involved in successful healing of soft tissues like ligaments and tendons to bone. Mesenchymal stem cells (MSCs) are multipotent cells found in tissue of mesodermal origin including bones, muscle, and fat. MSCs are able to differentiate into a variety of cell types including osteoblasts, chondrocytes, and tenocytes. They are commonly used for tissue engineering and regenerative medicine research and applications. Osteogenic (OS) media supplemented with the small molecules L-ascorbic acid 2-phosphate, beta-glycerophosphate (BGP), and dexamethasone has been shown to stimulate MSCs to differentiate into bone-forming osteoblasts. Certain method embodiments provided herein may optionally include coating suture with a mixture of fetal bovine serum (FBS) and OS to test the potential applicability for small molecule delivery as well. The ability to translate this suture coating technique beyond PRP to other growth factors or small molecules opens up numerous possibilities for regenerative medicine.

### DESCRIPTION OF ILLUSTRATED EMBODIMENTS

As shown in FIG. 1, a portion of a system 10 is shown wherein a first coating roller 12 and a second coating roller 14 are provided. Sutures 58 are being passed between the first roller 12 and second roller 14 to coat at least one side of the sutures 58. A first guide roller 16 is provided, and the sutures 58 are guided around the first guide roller such that the coated side of the sutures is not in contact with the first guide roller 16 and are allowed to dry such that the composition adheres onto the first side of the sutures.

FIG. 2 is a perspective view and FIG. 3 is a top view of another, non-limiting embodiment 20 of the system is shown, wherein a housing 24 is provided around the rollers. A first set of coating rollers 11 is shown with the first coating roller 12 and the second coating roller 14. A second set of coating rollers 26 is also provided, such that once the wound repair device, i.e., sutures, passes through the first set of coating rollers 11 to coat a first side of the wound repair device, and passes around the first guide roller 16 to dry the first side, the wound repair device may be passed through the second set of coating rollers 26 to coat the second side of the wound repair device with a composition (i.e., a biologic, a pharmaceutical composition, or a combination thereof). In one non-limiting embodiment, in the second set of coating rollers 26, the first roller 28 may not contain a composition, however, the second roller 30 may contain a composition, and the second side of the wound repair device may contact the second roller 30 as it passes through the second set of coating rollers 26. The wound repair device may then be passed around the second guide roller 32 such that the first side of the wound repair device contacts the second guide roller 32 to allow the coating on the second side of the wound repair device to dry. The embodiment 20 may include at least an entrance opening 34 and an exit opening 36 for the wound repair device to enter and exit the housing 24 of the system. In other further embodiments, additional sets of coating rollers may be used, and in some non-limiting embodiments, each set of coating rollers may be spaced between guide rollers to enable drying between each layer of coating applied to the wound repair device passed there between.

A vacuum, a suction hose, or other drying apparatus may be associated with the system 10 and 20, so as to add drying capabilities to the system and enable quicker drying of the wound repair device during use of the system, particularly when the system is use during a surgical procedure. As mentioned herein, vacuums are generally available in the OR, consequently, using the system embodiments described herein in the OR with the vacuums readily available, would allow this process to be easily integrated into an OR.

In some of the non-limiting embodiments described herein, the rollers may include any type of surface, including fluted surfaces, smooth surfaces, engraved surfaces, and all other types of surface treatments known to those skilled in the art. In some non-limiting embodiments, both rollers per set of coating rollers may include a textured, an engraved or a fluted surface, for example, which may contact one another in a way that is complementary during rotation of the rollers relative to one another during use of the system (i.e., during passage of the wound repair device to be coated).

FIGS. 4-6 show perspective views of an embodiment of a use of an intra-operative apparatus to coat a wound repair device in sequence. FIG. 4 is a perspective view of an embodiment of an intra-operative apparatus 40, having an enclosure 42, a coating chamber 46, a drying chamber 48, a chamber cover 50, and a frame 56 for holding or being associated with the wound repair device 58 to be coated. The apparatus 40 includes a first connector port 52 adjacent to the coating chamber 46, adapted to allow filling of the coating chamber 46 with a biologic or other substance, for example. A syringe or other device may be associated with the port 52, by luer lock or other method, and the biologic may be delivered to the coating chamber 46 in this manner in a non-limiting embodiment. The second connector port 54 is shown adjacent to the drying chamber 48. A drying device, suction tubing, an air hose, or other such device to provide a flow of air within the drying chamber 48 may be associated with the second connector port 54 on the apparatus 40. In FIG. 4, the wound repair device, i.e., sutures 58 are shown connected to the frame 56, which rests on ledge 45 of wall member 43, prior to being inserted into the coating chamber 46. The wall member 43 is rotatable related to enclosure 42. The chamber cover 50 is engaged to wall member 43 such that the chamber cover 50 rotates with the wall member 43. The two chambers are divided by a median portion 51 (see FIG. 7), in one non-limiting embodiment so as to prevent the biologic from entering the drying chamber, and the drying air from entering the coating chamber, for example.

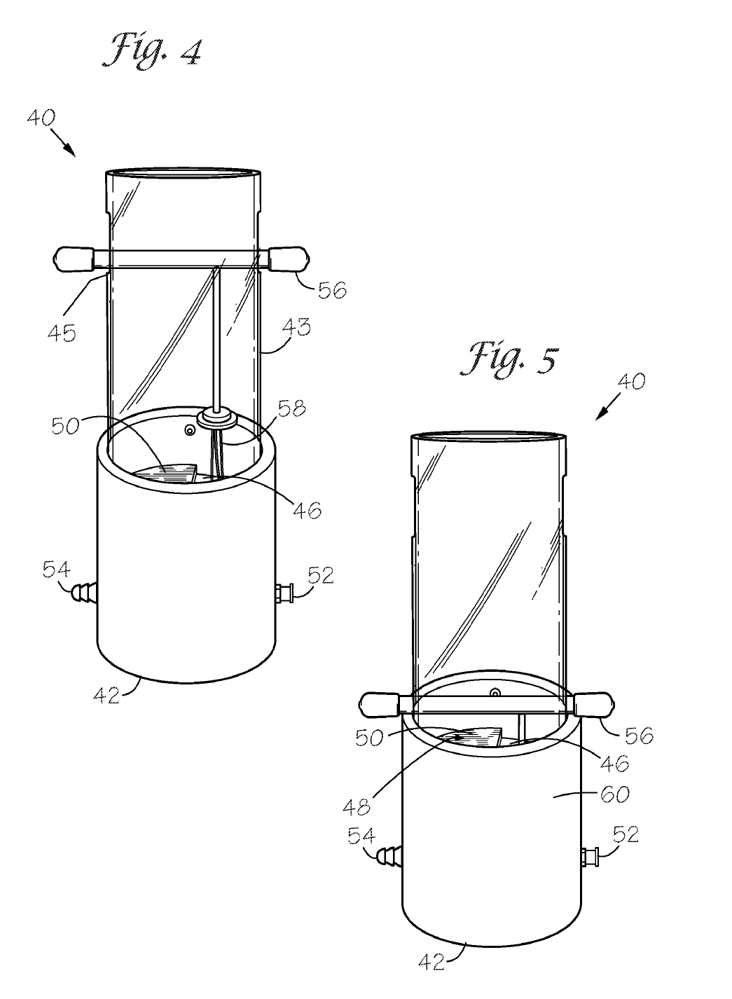

FIG. 5 is a perspective view of the apparatus 40, wherein the frame 56 has been adjusted such that the wound repair device 56 (not shown) has been inserted into the coating chamber 46, and into the biologic 60 there within to be coated. The frame 56 slides down slot 49 as shown in FIG. 7.

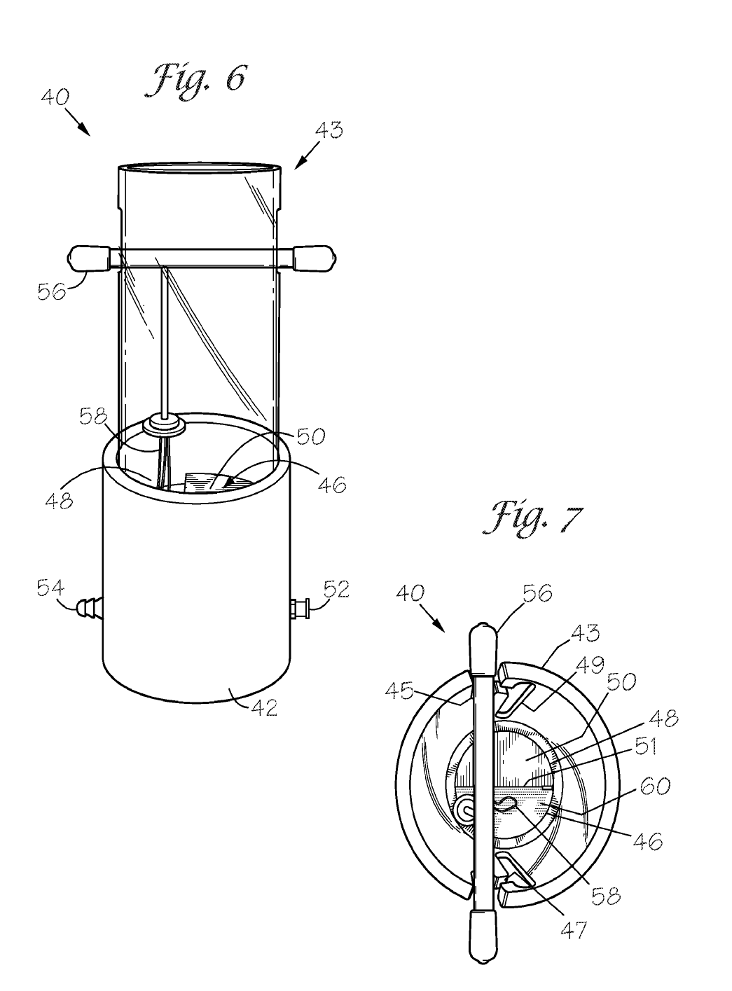

In the perspective view of FIG. 6, the apparatus 40 is shown wherein the wall member 43 has been rotated such that the wound repair device 58 held thereon has been removed from the coating chamber 46. and placed above the drying chamber 48. The frame 56 is held by a ledge 45 (see FIG. 7) defined in wall member 43 associated with the enclosure 42. The chamber cover 50 is shown as disposed over the coating chamber 46 in FIG. 6. Suction tubing or other such device (not shown in FIG. 6) may be connected to the second connector port 54 to effect the flow of air within the drying chamber 48.

FIG. 7 is a top view of the apparatus shown in FIG. 4, wherein the frame 56 is shown rested in the ledge 45 defined in wall member 43 holding the sutures above chambers. The chamber cover 50 is disposed over the drying chamber 48, and the sutures 58 are disposed over the coating chamber 46. During insertion, the frame is inserted through gap 47 into the slot 49 of the wall member 43, which allows the frame 56 to drop down to allow insertion of the sutures 58 into the biologic 60 contained in the coating chamber 46. As aforementioned, the apparatus and method embodiments provided herein include a rapid-drying process for coating layers of biologic on a wound repair device. The methods and apparatus embodiments may be used multiple times in sequence in order to provide a multi-layer coating on the wound repair device during a procedure, before use. Following coating of the sutures 58 with biologic 60, the frame is raised and placed back on the ledge 45. The wall member 43 is rotated 180 degrees and the cover 50 is shifted to cover chamber 46 and reveal drying chamber 48 such as that shown in FIG. 6. During drying of the sutures 58, it is not necessarily to lower the sutures 58 down into the drying chamber 48, since air flow will occur from the opening at the top of the wall member 43 through the drying chamber 48 and out connector 54. Consequently, the drying chamber 48 is not necessarily configured to receive the sutures 58. In an alternative embodiment, air pressure can be applied to connector 54 that flows through the drying chamber 48 and out the top of the wall member 43.

### EXAMPLES

### Materials and Methods

### Platelet Rich Plasma Preparation

One liter of porcine blood (Hollifield Farms, Covington, Ga.) was combined with 0.02 mg/ml of heparin sodium salt (VWR, Radnor, Pa.) to prevent coagulation. The whole blood was centrifuged as 50 ml aliquots at 40 g for 10 minutes. The plasma, buffy coat, and red blood cells were easily identified after centrifugation. Approximately 75% of the top portion of the plasma layer was removed, and the remaining platelet rich portion was carefully aspirated without disturbing the buffy coat layer.

### Suture Coating

Nine strands of non-expired 3-0 undyed braided vicryl 45 cm length sutures (Ethicon, Cincinnati, Ohio) underwent coating with PRP, and three sutures were used as uncoated negative controls. The three sutures in each experimental group underwent one, two, or four coating and drying cycles. Coating was performed using a custom fabricated dip tray and drying chamber. The entire length of the suture was submersed in the PRP for one second, and then placed in the drying chamber for five minutes. A convective airflow was obtained using standard wall suction. After coating, a 1 cm section was cut between the distal 30-31 cm of the suture and placed into 1 mL of distilled water for protein analysis. The custom drying chamber with suction tubing was used to produce a convective air flow to accelerate drying time.

### Coating Integrity Testing

The remaining 30 cm length of suture was threaded onto a free needle and passed through a 6.5 mm diameter fresh bovine extensor tendon (Animal Technologies, Tyler, Tex.) to simulate use during a surgical procedure (Figure II). A non-coated suture passed through the tendon serves as a control to determine how much protein would potentially be transferred from the tendon to the suture. Following passage through the tendon, a 1 cm section was cut between the middle 15 to 16 cm and placed into 1 mL of distilled water for protein analysis.

### Protein Quantification

Samples were agitated overnight to dissolve the PRP coating into solution. Protein quantity was measured using the Pierce BCA protein assay (Thermo Scientific, Grand Island, N.Y.) on samples prior to and after passage through the bovine tendon. The three data points in each experimental group were averaged for statistical analysis.

### Tensile Testing

After coating, drying, and passing through the tendon, the remaining 15 cm of suture underwent tensile stress using a Instron 3345 universal testing system (Instron, Norwood, Mass.) until failure. The program parameters were an initial strain rate of 0.25 mm/sec until a preload of 10N was reached, and then followed by a strain rate of 1 mm/sec until failure. The tensile stress at failure was recorded for each suture and the average calculated for statistical analysis.

### Statistical Analysis

Results from the BCA assay were analyzed via a 2×4 ANOVA followed by a Tukey's post-hoc test. Tensile testing data was analyzed using a one-way ANOVA followed by a Tukey's post-hoc test.

### RESULTS

### Protein Coating Quantification

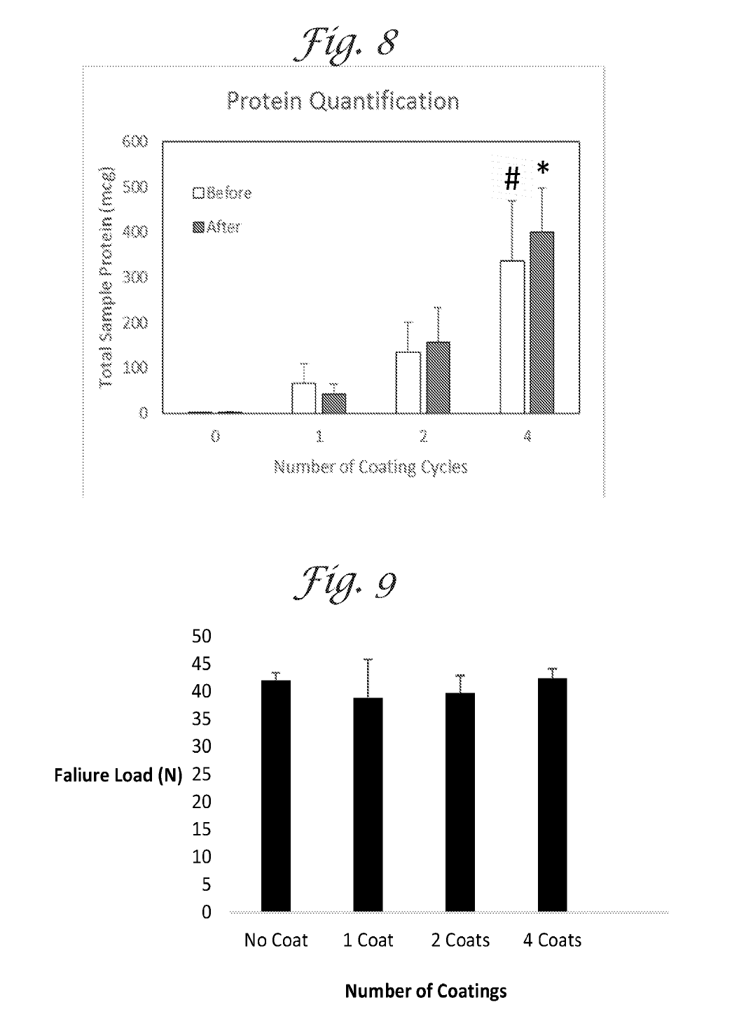

The total protein content in each 1 cm section of suture showed a directly proportional trend with respect to the number of coating and drying cycles (FIG. 8). This trend approached statistical significance (p=0.06) for the suture before tendon passage, and was statistically significant (p=0.03) for the suture after tendon passage. There was no trend or statistical difference when comparing protein content of the coating before and after tendon passage. This indicates the coating is stable and is not stripped off during standard handling and suturing.

FIG. 8 provides a graphical representation of total protein content of each sample compared to the number of coating cycles. The measurements were made both before and after passage of the suture through a tendon. (#): p=0.06 when comparing before 0 or before 1 vs before 4; (*): p=0.03 when comparing after 0 or after 1 vs after 4.

### Tensile Testing

The tensile load to failure showed no significant difference with relation to the number of PRP coatings (FIG. 9). This indicates that the PRP coating does not affect the mechanical integrity of the suture. FIG. 9 provides a graphical illustration showing tensile testing to failure showed no significant effect due to the PRP coating process.

The methods, systems and apparatus embodiments provided herein for coating sutures with a biologic and/or pharmaceutical composition, such as, for example, PRP or plasma may also, or may alternatively be useful as a method for delivery of pharmaceuticals to a target patient location. The wound repair devices described herein may be coated with pharmaceuticals and targeted to a location to promote healing and/or a whole host of other treatment methods. The pharmaceuticals can be added to a coating solution (including PRP, plasma or other protein or carbohydrate based solution that will adhere to the suture when dried) at a desired concentration. The coating and drying sequence can then be performed as described in embodiments herein so as to coat the wound repair device (i.e., suture) with the composition including the pharmaceutical. The wound repair device may also be coated with a pharmaceutical solution, (without PRP, plasma, or other protein or carbohydrate), in another non-limiting embodiment. The embodiments described herein will provide enhanced local concentrations of pharmaceuticals, decreasing the potential for systemic side effects of medications provided oral or parenterally, for example, to reduce potential kidney or liver toxicity.

It should be borne in mind that all patents, patent applications, patent publications, technical publications, scientific publications, and other references referenced herein are hereby incorporated by reference in this application in order to more fully describe the state of the art to which the present invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise.

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

What is claimed is:

1. A system for applying a composition to a wound repair device for tissue repair, comprising:
a housing comprising the following associated therein:
at least a first set of coating rollers comprising at least a first roller and a second roller, configured to receive a wound repair device there between, said first and second roller configured to rotate in opposing directions as the wound repair device is passed there between, wherein the first and/or second roller comprises the composition to be applied to the wound repair device, such that when the wound repair device is passed between the first and second rollers, the composition is applied to the wound repair device; and at least a first guide roller configured to rotate and guide the wound repair device to allow the composition to adhere onto the wound repair device, such that when the wound repair device is guided by the first guide roller, the composition is dried thereon.

2. The system of claim 1 further comprising at least a second set of coating rollers, comprising a first roller and a second roller configured to receive the wound repair device from the first guide roller, the second set of coating rollers rotate in opposing directions as the wound repair device is passed there between, wherein at least one roller of the second set of rollers comprises the composition, such that when the wound repair device is passed between the second set of rollers, the composition is applied to the wound repair device.

3. The system of claim 2, further comprising a second guide roller configured to guide the wound repair device and allow the composition to adhere onto the wound repair device, such that when the wound repair device is guided by the second guide roller, the composition is dried thereon.

4. The system of claim 1, wherein the composition includes a biologic, a pharmaceutical composition, or a combination thereof.

5. The system of claim 1, wherein the composition on the first and/or second roller of the first set of coating rollers is different from the composition of the first and/or second roller of the second set of coating rollers.

6. The system of claim 4, wherein the biologic comprises plasma.

7. The system of claim 6, wherein the plasma comprises a platelet rich plasma (PRP).

8. The system of claim 1, wherein the wound repair device comprises one or more sutures.

9. The system of claim 4, wherein the composition comprises a pharmaceutical composition.

10. The system of claim 1, further comprising a vacuum associated therewith to vacuum dry the composition onto the wound repair device.

11. The system of claim 1, comprising a first opening in the housing configured to allow the wound repair device to enter the housing, and a second opening in the housing configured to allow the wound repair device to exit the housing.

12. The system of claim 1, wherein at least the first and/or second roller of the first set of coating rollers comprises an engraved surface.

13. The system of claim 2, wherein at least the first and/or second roller of the second set of coating rollers comprises an engraved surface.

14. The system of claim 2, wherein the first roller of the first set of coating rollers and the second roller of the second set of coating rollers comprises the composition, such that when the wound repair device is passed between the first set of coating rollers, a first side of the wound repair device contacts the first roller and is coated with the composition, and when the wound repair device is passed between the second set of coating rollers, a second side of the wound repair device contacts the second roller and is coated with the composition.

15. The system of claim 14, wherein when the wound repair device is guided via the first guide roller, the first side of the wound repair device is dried.

16. The system of claim 14, wherein when the wound repair device is guided via the second guide roller, the second side of the wound repair device is dried.

17. The system of claim 1, wherein the first set of coating rollers rotate relative to one another to apply a pressure to the wound repair device as it passes between the first set of coating rollers.

18. The system of claim 2, wherein the second set of coating rollers rotate relative to one another to apply a pressure to the wound repair device as it passes between the second set of coating rollers.

19. A method of coating a wound repair device with a composition, comprising:
threading a wound repair device between a first set of guiding rollers comprising a first roller and a second roller, such that the first and second rollers contact a first side and a second side of the wound repair device, respectively, wherein the first roller comprises a first composition thereon, such that the first side of the wound repair device is coated with the first composition;
guiding the wound repair device around a first guide roller, such that the second side of the wound repair device contacts the guide roller, allowing the first side of the wound repair device to dry;
threading the wound repair device between a second set of coating rollers comprising a first roller and a second roller, such that the first roller and second roller contacts the first side and the second side of the wound repair device, respectively, wherein the second roller comprises a second composition thereon, such that the second side of the wound repair device is coated with the second composition as the wound repair device is threaded through the second set of coating rollers; and
guiding the wound repair device around a second guide roller, such that the first side of the wound repair device contacts the guide roller, allowing the second side of the wound repair device to dry.

20. The method of claim 19, further comprising associating a vacuum with the wound repair device to facilitate the drying of the composition on the wound repair device.

21. The method of claim 19, wherein the wound repair device comprises sutures.

22. The method of claim 19, wherein the composition comprises a biologic, a pharmaceutical composition, or a combination thereof.

23. The method of claim 22, wherein the biologic comprises a plasma coating.

24. The method of claim 23, wherein the plasma coating comprises a platelet rich plasma.

25. The method of claim 19, wherein the first composition and the second composition are chemically equivalent.

26. The system of claim 4, wherein the biologic is obtained from a subject, and further comprising threading the wound repair device into a tissue of the subject from which the biologic was obtained.

27. The system of claim 4, wherein the biologic is obtained from an allogenic, xenogenic, or autogenic source.

28. A method comprising
obtaining an isolated platelet rich plasma (PRP) sample; and
applying the sample to a wound repair device under conditions to provide a dry coat of PRP onto the wound repair device.

29. The method of claim 28, wherein the PRP is obtained from a subject, and further comprising administering the wound repair device comprising a dry coat of PRP into or onto a tissue of the subject from which the PRP was obtained.

30. The method of claim 28, wherein the PRP is obtained from an allogenic source.

31. The method of claim 28, wherein the PRP is obtained from a xenogenic source.

32. The method of claim 28, wherein the PRP is obtained from an autogenic source.

33. The method of claim 28, wherein the wound repair device is a suture.

34. A suture produced by the method of claim 33.

35. A method comprising threading the suture of claim 34 into a tissue of a subject in need.

36. A rapid intra-operative coating method, comprising:
obtaining a biologic;
obtaining a wound repair device; and
coating the wound repair device with the biologic under conditions to enhance drying of the biologic onto the wound repair device.

37. The rapid intra-operative coating method of claim 36, wherein the biologic is obtained from an allogenic source, a xenogenic source, or an autogenic source.

38. The rapid intra-operative coating method of claim 36, wherein the biologic is an isolated platelet rich plasma sample (PRP).

39. The rapid intra-operative coating method of claim 36, wherein the biologic comprises a solution including one or more of: a protein, lipid and/or carbohydrate.

40. The rapid intra-operative coating method of claim 36, wherein the wound repair device comprises a sponge, a bandage, beads, a patch or a suture.

41. The rapid intra-operative coating method of claim 36, wherein the conditions to enhance drying of the biologic onto the wound repair device comprises delivering a convective flow of air to the wound repair device in a contained chamber.

42. The rapid intra-operative coating method of claim 41, comprising a vacuum to deliver the convective flow of air to the wound repair device.

43. The rapid intra-operative coating method of claim 41, further comprising adding a pharmaceutical composition to the biologic prior to coating.

44. The rapid intra-operative coating method of claim 36, wherein following coating the wound repair device, a vacuum is applied to the wound repair device to dry the biologic onto the wound repair device.

45. The rapid intra-operative coating method of claim 43, wherein coating comprises applying the wound repair device with the biologic while in a chamber.

46. The rapid intra-operative coating method of claim 36, further comprising repeating the applying step to provide a subsequent layer of the biologic onto the wound repair device.

47. The rapid intra-operative coating method of claim 46, further comprising associating a vacuum with the wound repair device to dry the subsequent layer of biologic onto the wound repair device.

48. The rapid intra-operative coating method of claim 36, wherein obtaining the biologic and coating steps are conducted within 2 hours preceding or during an operative procedure on a patient.

49. An intra-operative apparatus for coating a wound repair device, comprising:
at least one coating chamber; and
at least one drying chamber;
wherein the coating chamber is configured to contain a composition to be coated on a wound repair device inserted therein, and the drying chamber is configured to dry the composition-coated wound repair device.

50. The intra-operative apparatus for coating a wound repair device of claim 49, wherein the composition comprises a biologic, a pharmaceutical composition, or a combination thereof.

51. The intra-operative apparatus for coating a wound repair device of claim 49, comprising a first connector port in communication with the coating chamber, wherein a composition may be introduced through the first connector port.

52. The intra-operative apparatus for coating a wound repair device of claim 51, wherein the first connector port is adapted to receive a syringe, said syringe comprising the composition to be introduced into the coating chamber.

53. The intra-operative apparatus for coating a wound repair device of claim 49, comprising a second connector port in communication with the drying chamber, the second connector port configured to connect to a suction device or an airflow device, wherein the suction or airflow device is configured to provide a movement of air in the drying chamber.

54. The intra-operative apparatus for coating a wound repair device of claim 53, wherein the suction device provides a convective flow of air in the drying chamber when associated therewith to dry the composition on the wound repair device.

55. The intra-operative apparatus for coating a wound repair device of claim 53, further comprising an airflow device connected to the second connector port.

56. The intra-operative apparatus for coating a wound repair device of claim 55, further comprising a chamber cover that is shiftable between the coating chamber and the drying chamber to cover the chamber that is not in use.

57. The intra-operative apparatus for coating a wound repair device of claim 49, wherein the wound repair device may be shifted from the coating chamber and the drying chamber.

58. The intra-operative apparatus for coating a wound repair device of claim 57, further comprising a frame configured to associate with the wound repair device to be coated, wherein the frame can be manipulated by a user relative to the coating chamber and the drying chamber to insert the wound repair device to be coated in the respective chamber.

59. The intra-operative apparatus for coating a wound repair device of claim 49, wherein the wound repair device comprises a suture, a sponge, a bandage, a patch, or a polymer bead.

60. An intra-operative system for coating a wound repair device, comprising:
a coating chamber for containing a composition; and
a drying chamber for drying the composition on a wound repair device, wherein the coating chamber and the drying chamber each comprise an opening to receive a wound repair device to be coated, and wherein the wound repair device to be coated with composition is inserted into the coating chamber, and is subsequently inserted into the drying chamber to dry the composition onto the wound repair device.

61. The intra-operative system for coating a wound repair device of claim 60, wherein the coating chamber and the drying chamber are contained within an enclosure.

62. The intra-operative system of claim 60, wherein the coating chamber comprises a coating chamber aperture for receiving a composition.

63. The intra-operative system of claim 60, wherein the drying chamber comprises a drying chamber aperture for associating with a device to provide a flow of air within the drying chamber to dry the composition onto the wound repair device placed therein.

64. The intra-operative system of claim 63, comprising a suction device configured to associate with the drying chamber aperture to provide a convective flow of air over the wound repair device to dry the composition on the wound repair device.

65. The intra-operative system of claim 64, comprising an airflow device configured to associate with the drying chamber aperture to deliver air to the drying chamber to dry the composition on the wound repair device.

66. A method comprising:
obtaining a composition comprising: a biologic, a pharmaceutical composition, or a combination thereof; and
applying the composition to a wound repair device under conditions to provide a dry coat of the composition onto the wound repair device.

67. A wound repair device produced by the method of claim 66.

68. The method of claim 66, wherein the biologic comprises PRP.

69. The method of claim 66, wherein the composition comprises mesenchymal stem cells (MSCs) orosteogenic media (OS), or both.

70. The method of claim 66, wherein the composition comprises ascorbic acid, beta-glycerophosphate (BGP), and dexamethasone.

71. A method for coating a wound repair device, comprising:
obtaining an apparatus of claim 49 or 60;
inserting the wound repair device into the coating chamber, the coating chamber comprising a composition to be applied to the wound repair device;
removing the wound repair device from the coating chamber; and
inserting the wound repair device into the drying chamber to dry the composition onto the wound repair device.

72. The method of claim 71, further comprising affixing the wound repair device onto a frame device for insertion of the wound repair device into the coating and/or drying chambers by movement of the frame relative to the chambers.

73. The method of claim 71, wherein following drying the composition onto the wound repair device, inserting the wound repair device into the coating chamber to provide at least one additional coating of the composition onto the device.

74. The method of claim 73, further comprising inserting the wound repair device into the drying chamber to dry the at least one additional coating.
